(19) United States
(12) Patent Application Publication
(10) Pub. No.: US 2022/0000881 A1
(43) Pub. Date: Jan. 6, 2022

(54) TREATMENT OF EXPOSURE TO CHLORINE GAS WITH SCOPOLAMINE

(71) Applicant: REPURPOSED THERAPEUTICS, INC. DBA DEFENDER, Tampa, FL (US)

(72) Inventor: David Helton, Dana Point, CA (US)

(21) Appl. No.: 17/289,711

(22) PCT Filed: Oct. 31, 2019
(86) PCT No.: PCT/US2019/059272

§ 371 Date: Apr. 28, 2021

Related U.S. Application Data
(60) Provisional application No. 62/753,270, filed on Oct. 31, 2018.

(51) Int. Cl. A61K 31/5386; A61P 39/02

(52) U.S. Cl. CPC A61K 31/5386; A61P 39/02

(57) ABSTRACT

Exposure of a subject to chlorine gas is treated by administering scopolamine to the subject.

### CROSS-REFERENCE TO RELATED APPLICATIONS

This application claims the benefit of priority under 35 U.S.C. § 119(e) from U.S. Patent Application No. 62/753,270 filed on Oct. 31, 2018. The disclosure of the foregoing application is incorporated herein by reference in its entirety.

### BACKGROUND

Chlorine gas poisoning can result from exposure to chlorine gas (Cl₂). Acute chlorine poisoning affects the respiratory system and can cause coughing and difficulty breathing, as well as skin irritation or chemical burns. Chlorine gas poisoning can also result in eye irritation, nausea, vomiting, and/or a headache. Common domestic exposures result from the mixing of chlorine bleach with acidic washing agents or as a result of the chlorination of water.

Chlorine gas has also been used in warfare, and at sufficiently high levels exposure to chlorine gas can be fatal. Because Cl₂ is easily manufactured, its use as a chemical weapon is difficult to control. There is therefore a continuing need for improved treatments for chlorine gas exposure.

### SUMMARY

Chlorine exposure and inhalation at high doses results in morbidity, including abnormal epileptiform discharges, and can result in death. Administration of scopolamine to subjects after chlorine exposure is effective at improving survival, hypotonia, and bradycardia, and in minimizing ataxia/seizure activity.

The present invention includes a pharmaceutical composition comprising scopolamine for use in the treatment of exposure to chlorine gas. The composition can be formulated in an amount which delivers between 0.01 mg/kg and 0.3 mg/kg scopolamine to a subject in need thereof, for example between 0.05 mg/kg and 0.2 mg/kg or between 0.1 mg/kg and 0.15 mg/kg. In addition, the composition can be formulated in an amount which delivers a total dose of between 0.2 mg and 10 mg, between 0.5 mg and 4 mg, or between 1 mg and 2 mg.

The scopolamine composition can be formulated for administration intranasally, intravenously, intratracheally, intramuscularly, by inhalation, or in other ways known to the art. In one example, the composition is an intranasal formulation in a dose of between 0.2 mg and 2 mg scopolamine, such as a dose of 0.2 mg or 0.4 mg scopolamine. The scopolamine compound can be scopolamine HBr.

The scopolamine composition can be administered either prior to or following exposure of a subject to chlorine gas. For example, the composition can be administered within 10 hours prior to exposure to chlorine gas, preferably less than 5 hours, less than 3 hours, or less than 1 hour prior to exposure. The composition can be administered a plurality of times following exposure to chlorine gas, in order to maintain a subject's blood levels of scopolamine.

In one embodiment, the scopolamine composition can be provided in a kit that further includes a delivery device, such as an inhaler, syringe, or intranasal spray device.

In a further embodiment, the present invention comprises a method of treating exposure to chlorine gas by administering a therapeutically effective amount of a pharmaceutical composition comprising scopolamine to a subject in need thereof. The subject can have a medical condition selected from the group consisting of pulmonary edema, tracheobronchitis, and acute respiratory distress syndrome, for example.

### FIGURES

FIG. 1 is a chart showing the survival of subjects treated with scopolamine following high dose Cl₂ exposure.

FIG. 2 is a chart showing blood levels of carbon dioxide in subjects exposed to chlorine gas, including subjects treated with scopolamine.

FIG. 3 is a chart showing the percent of subjects experiencing tremors or seizures following exposure to Cl₂, including subjects treated with scopolamine.

### DESCRIPTION

### Definitions

As used herein, the following terms and variations thereof have the meanings given below, unless a different meaning is clearly intended by the context in which such term is used.

“About” when used in reference to a numerical value means plus or minus ten percent of the indicated amount. For example and not by way of limitation, “about 10” means between 9 and 11, and “about 10%” means between 9% and 11%.

“Chlorine” and “chlorine gas” as used herein generally refers to the compound Cl₂, which is a gas at room temperature (having a boiling point of −34° C.). The present treatments for chlorine exposure can also be applied to exposure to other reactive chlorine-containing compounds, such as methyl chloride (CH₃Cl), chloroform (CHCl₃), methylchloroform (CH₃CCl₃), phosgene (COCl₂), dichloromethane (CH₂Cl₂), chlorinated ethylenes (C₂HCl₃, C₂Cl₄), chlorinated ethanes (CH₄Cl₂, C₂H₂Cl₄), and inorganic chlorine gases such as hydrogen chloride (HCl).

“Gel” refers to a composition in which the constituents are present in a viscous liquid or in a true gel, i.e. a cross-linked system in which liquid particles are dispersed in a solid medium.

“Medical condition” refers to conditions which cause disease, discomfort and/or disability in a subject.

“Pharmaceutical effect” refers to an effect in restoring, correcting or modifying a physiological function of a subject, including the cure, mitigation, treatment or prevention of a medical condition in the subject. A “pharmaceutical composition” and a “medicament” are compositions having a pharmaceutical effect.

“Pharmaceutically acceptable salt” refers to a compound derived from the chemical reaction of an acid or base with a parent compound, and which is safe and effective for use with humans and other subjects. For example, a scopolamine salt can be produced by reacting scopolamine with various acids such as hydrochloric acid, hydrobromic acid, hydroiodic acid, nitric acid, phosphoric acid, sulfuric acid, and the like. Pharmaceutically acceptable salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate salts.

“Scopolamine” refers to the compound (−)-(S)-3-Hydroxy-2-phenylpropionic acid (1R,2R,4S,7S,9S)-9-methyl-3-oxa-9-azatricyclo[3.3.1.02,4]non-7-yl ester (represented by the formula C₁₇H₂₁NO₄) and pharmaceutically acceptable salts thereof, such as scopolamine hydrobromide and scopolamine hydrobromide trihydrate. Scopolamine has the following structure:

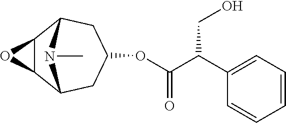

“Scopolamine analog(s)” refers to compounds that have the same backbone as scopolamine, but in which one or more moieties have been substituted by, or replaced with, other substituents or moieties. Such substitutions or replacements are in accordance with the permitted valence of the substituted atom and the substituent and result in a stable compound, i.e., a compound that does not spontaneously undergo transformation under conditions of storage and use of the present formulation. It is to be understood that any and all known substituents or moieties of organic compounds can be used. In a broad aspect, the permissible substituents include acyclic and cyclic, branched and unbranched, carbocyclic and heterocyclic, aromatic and nonaromatic substituents of organic compounds.

“Subject” refers to an individual treated with a pharmaceutical composition as described herein. The subject is preferably a mammal, and more preferably is human.

“Suitable for intranasal administration” refers to any mode of administration of a medicament (i.e., a composition comprising scopolamine) intranasally, i.e., into the nose of a subject.

“Therapeutically effective amount” refers to the amount of a composition, or amount of scopolamine or analog thereof, that, when administered to a subject, is sufficient to have an effect in restoring, correcting, or modifying a physiological function of a subject, including in treatment of a medical condition associated with exposure to chlorine.

“Treatment,” with respect to the exposure of a subject to chlorine, refers to a medical intervention which attenuates, prevents, and/or counteracts the effects of such exposure. Treatments can refer to the prophylactic administration of the present compounds and compositions to subjects at risk of exposure to chlorine prior to an anticipated exposure, and/or can refer to the administration of the present compounds and compositions following such exposure.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise. Ranges which are described as being “between” two values include the indicated values.

### Scopolamine

Scopolamine is a muscarinic antagonist structurally similar to the neurotransmitter acetylcholine and can act by blocking the muscarinic acetylcholine receptors. Blocking acetylcholine from binding to its receptors blocks acetylcholine-mediated nerve impulses from travelling through the body. It is thus classified as an anticholinergic agent.

Scopolamine is also referred to as hyoscine and scopine tropate. The scopolamine used in the present invention can be the compound scopolamine itself, a scopolamine salt, an analog of scopolamine, or mixtures thereof. A preferred salt is scopolamine hydrobromide (scopolamine HBr). Other suitable scopolamine compounds that can be used in the compositions and methods of the present invention include, but are not limited to scopolamine hydrobromide trihydrate, scopolamine hydrochloride, scopolamine methyl nitrate, methscopolamine nitrate, scopolamine methyl bromide, scopolamine hydrobromide hydrate, scopolamine bromide, scopolaminium bromide, and the like.

### Formulation

The scopolamine of the present invention is formulated as a pharmaceutical composition for administration in ways known to the art, including intramuscular, intravenous, subdermal, intratracheal, and intranasal administration and administration by inhalation. Depending on the route of administration, the scopolamine composition can be formulated as a liquid, as a powder or other solid, or as a gel. The pharmaceutical composition can include one or more pharmaceutically acceptable carriers and/or other pharmaceutically acceptable excipients, for example to stabilize and/or deliver the composition to a subject. Excipients for the present pharmaceutical composition can include appropriate additives such as pharmaceutically effective carriers (i.e., sterile water, water, saline, and the like), buffers, neutralizing agents, stabilizers, humectants, viscosity builders, chemical stabilizers, thickeners, diluents, and/or solvents. Examples of excipients for some embodiments include, but are not limited to, alcohols and polyglycols, glycerin, waxes, water, deionized water, fatty acid esters, and the like, mixtures thereof and combinations thereof.

Additives that can be included in the present formulation include components with beneficial properties in connection with the use of the present formulation. For example, components that sooth or protect the nasal mucosa such as aloe or a nasal moisturizer can be included in formulations for nasal administration. A component such as caffeine which enhances absorption of other components of the formulation can also be included. In addition, components which confer other beneficial properties to a subject, such as vitamins, can be included in the formulation.

For intranasal administration, the composition preferably includes one or more gelling agents such as acacia, alginic acid, bentonite, Carbopols (carbomers), carboxymethyl cellulose, ethylcellulose, gelatin, hydroxyethyl cellulose, hydroxypropyl cellulose, magnesium aluminum silicate (Veegum), methylcellulose, poloxamers (Pluronics), polyvinyl alcohol, sodium alginate, tragacanth, and xanthan gum. Such gels can be either a true gel or a viscous liquid. When a viscous liquid is used, the viscosity of the liquid is preferably between 1,000 and 3,000 centistokes (square millimeters per second), and more preferably about 1,800 centistokes, though more viscous liquids can also advantageously be used, such as liquids of 5,000 centistokes, 10,000 centistokes, or more. The formulated composition preferably has a Bloom strength of less than about 125 Bloom, with Bloom strength being the weight in grams needed by a specified plunger (normally with a diameter of 0.5 inch) to depress the surface of the gel by 4 mm without breaking it at a specified temperature, typically 10° C. More preferably, the Bloom strength is between 15 and 100, or between 25 and 85, or between 35 and 60. The gel has the capability to adsorb or stick to the inner lining of the nasal cavity of a subject so that at least the active ingredient or ingredients of the formulation can be absorbed.

Compositions for nasal administration can advantageously further include buffering agents and preservatives. Examples of buffering agents include, but are not limited to, sodium citrate, phosphate buffer, sodium salts of various acids, and the like. Compositions with sodium citrate can have a pH of less than about 5, such as a pH of between 3 and 4, for example a pH of between about 3.4 and 3.6 In other embodiments, the buffering agent can be omitted from the composition. Preservatives such as benzalkonium chloride, parabens, quarternary ammonium compounds, aryl acids, aryl alcohols, alkyl acids, thiomersal, and antimicrobial agents can be used, for example.

### Manufacturing Methods

The scopolamine compositions can be manufactured by methods known in the art, for example by combining, mixing, and/or compounding ingredients identified herein. The compounds disclosed herein, including scopolamine and analogs thereof, can be obtained commercially or can be synthesized using techniques generally known to those of skill in the art. The starting materials and reagents used in preparing these compounds are either available from commercial suppliers such as Sigma-Aldrich Chemical Co. and Fisher Scientific, or can be prepared by methods known in the art.

The scopolamine compositions of the present invention can be provided in the form of a kit, for example a kit for use in a combat situation. The kit can include a composition comprising scopolamine or an analog thereof, a delivery device such as an inhaler, syringe, or intranasal spray device or other applicator, and instructions for using the composition and the delivery device. In some embodiments the scopolamine composition will be in powder or other dry form and will need to be dissolved or emulsified in a liquid, such as water for injection or deionized water, in which case the kit preferably includes this.

### Administration

Scopolamine compositions can be administered to subjects in ways known to the art, such as via intramuscular, intravenous, subdermal, intratracheal, and intranasal administration and administration by inhalation. Other methods for delivering scopolamine to a subject's bloodstream and/or to a site of contact with chlorine gas (such as the trachea and lungs) can also be used.

In one embodiment, a scopolamine composition can be administered intranasally in the form of a gel. In order to administer a desired amount of gel, the gel can be administered with a metered dispenser device adapted to dispense the desired amount of gel. In some uses, the device is adjustable in order to administer different, predetermined, metered amounts of the gel as needed. The device can be either a single-use or multiple-use device. The gel can also be packaged in single-use ampules or other containers such as containers made with a blow-fill-seal process. In this case, such containers can retain either the desired amount of gel for a single administration, or can retain gel in smaller units of administration so that different doses can be administered through the use of a combination of ampules or other containers.

### Treatment of Chlorine Exposure

Exposure to chlorine gas can severely damage the airway mucosa of a subject, in addition to other effects. Hydrogen chloride (HCl) and oxygen free radicals (hypochlorous, HOCl) are liberated when Cl₂ contacts water in the upper and lower respiratory tract. Acute inflammation of the conjunctiva, nose, pharynx, larynx, trachea, and bronchi are some of the immediate effects of chlorine gas toxicity. Local edema secondary to active arterial and capillary hyperemia can develop due to irritation of the airway mucosa, leading to filling of the alveoli with edema fluid, resulting in pulmonary congestion and dose-dependent epithelial cell injury. At low levels of exposure the upper airways and eyes irritated, but as the levels of exposure increase the nasopharynx and larynx can be injured. Exposure to chlorine can further result in tracheobronchitis (inflammation of the trachea and bronchi) and/or acute respiratory distress syndrome, in which fluid fills up the lungs' air sacs resulting in an increase the amount of carbon dioxide in the bloodstream. Pulmonary edema develops within 6 to 24 hours of higher exposures.

Exposure to high levels of chlorine (Cl₂) gas can cause death within hours of inhalation. In rats, high levels of chlorine causes hypoxemia, acidosis, low cardiac output (CO), and associated respiratory distress. Neurobehavioral functions have been shown to be impaired after chlorine exposure, and chlorine inhalation at high doses can cause abnormal epileptiform discharges, including signs of hyperexcitability, spike and waves associated with myoclonic jerks, and repetitive periodic lateral epileptiform discharges (PLED). Epileptiform discharges can progress over time and result in death.

Scopolamine is effective at improving survival, hypotonia, bradycardia, and in minimizing ataxia/seizure activity, following exposure to chlorine, especially Cl₂. Scopolamine or a scopolamine compound is preferably administered either prior to chlorine exposure or as soon thereafter as possible. Scopolamine has a half-life on the order of between 8 and 9.5 hours, so it can be administered up to 10 hours prior to exposure, more preferably less than 9 hours, less than 8 hours, less than 7 hours, less than 6 hours, less than 5 hours, less than 4 hours, less than 3 hours, less than 2 hours, or less than 1 hour prior to exposure to chlorine gas. Treatment following chlorine exposure is preferably commenced within about 5 minutes, 10 minutes, 15 minutes, 30 minutes, 1 hour, 2 hours, 6 hours, or longer following chlorine gas exposure. A scopolamine composition can be administered once following exposure to chlorine gas and discontinued if clinical improvement occurs, or can be administered a plurality of times over the course of time following exposure. One of ordinary skill in the art can adjust the frequency of administration according to methods known in the art.

Scopolamine or a scopolamine compound can be administered in doses of between 0.005 mg/kg and 0.5 mg/kg (i.e., mg scopolamine per kilogram of the subject), between 0.01 mg/kg and 0.3 mg/kg, between 0.05 mg/kg and 0.2 mg/kg, or between 0.1 mg/kg and 0.15 mg/kg, for example. Total doses of 10 mg or less, such as 4 mg, 2 mg, 1 mg, 0.5 mg, or 0.2 mg, are preferred, in order to avoid side effects or adverse events caused by the scopolamine concentration.

In one embodiment, the gel is administered intranasally in metered doses of between 0.2 mg and 2 mg scopolamine HBr per dose. For example, a dose of 0.4 mg scopolamine can be administered by applying one 0.2 mg metered dose into each nostril, while a dose of 0.6 mg can be administered by applying three 0.2 mg metered doses, or 0.8 mg can be administered by applying four 0.2 mg metered doses. When more than two metered doses are administered, they are preferably administered into alternating nostrils. Doses can be administered in immediate succession or can be spaced apart, such as by waiting 20 minutes between metered doses. For certain uses, doses can be administered from hours to days between doings. In one embodiment the pharmaceutical composition can include less than about 0.5 percent, less than about 0.3 percent, or less than about 0.2 percent by weight scopolamine or an analog thereof, such as about 0.15 percent by weight scopolamine or an analog thereof.

### EXAMPLES

### Example 1—Formulation

A 0.12 gram dose of an intranasal gel formulation of scopolamine can be formulated as follows:

| Component | Amount |
|---|---|
| scopolamine HBr | 0.20 mg |
| sodium citrate (buffer) | 0.42 mg |
| citric acid | 0.89 mg |
| sodium metabisulfite | 0.12 mg |
| glycerin | 6.00 mg |
| benzalkonium chloride | 0.05 mg |
| polyvinyl alcohol | 12.0 mg |
| purified water | q.s. |

### Example 2—Formulation

A 0.12 gram dose of another intranasal gel formulation of scopolamine can be formulated as follows:

| Component | Amount | |
|---|---|---|
| scopolamine HBR | 0.2 | mg |
| sodium Citrate (buffer) | 0.34 | mg |
| citric acid | 0.73 | mg |
| sodium metabisulfite | 0.1 | mg |
| glycerin | 5.0 | mg |
| benzalkonium chloride | 0.040 | mg |
| polyvinyl alcohol | 10 | mg |

| | |
|---|---|
| purified water | 0.10 |

### Example 3—Manufacturing Method

In order to produce the formulation of Example 2 above, a 10 kg batch can be produced as follows. A portion of purified water is placed in a 3 L stainless steel vessel and heated to 25° C. on a hot plate fitted with a 2.7 inch stainless steel propeller. The water is stirred at 200 rpm. Sodium citrate is added to the vessel and stirred to dissolve it (200 rpm for 5 minutes). Citric acid (anhydrous) is then added to the vessel and stirred to dissolve it (200 rpm for 5 minutes), after which sodium metabisulfite is added to the vessel and stirred to dissolve it (200 rpm for 5 minutes). Scopolamine HBr is then added to the vessel and stirred to dissolve it (200 rpm for 10 minutes), after which glycerin is added to the vessel and stirred to dissolve it (200 rpm for 10 minutes). Finally, a benzalkonium chloride solution is added to the vessel and stirred to dissolve it (200 rpm for 10 minutes).

Another portion of water is placed in a 20 L stainless steel vessel fitted with a stainless steel propeller. The vessel is placed on a hot plate with heat setting on “OFF”. With stirring at 150 rpm, polyvinyl alcohol is added slowly to the vessel over a period of about 25 minutes. During dispersion, the stirring speed is increased to 175 rpm. The dispersion is heated to 75° C. and maintained at this temperature for 30 minutes while stirring. Then, the hot plate is removed and stirring continues until the dispersion cools down to about 25° C. The mixture in the 3 L stainless steel vessel is then added to the 20 L vessel while stirring at 400 rpm for 30 minutes.

### Example 4: Treatment of Chlorine Exposure

In a study of the effectiveness of scopolamine in treating chlorine exposure, Sprague-Dawley rats (290-340 g, male) were exposed for 30 minutes to 550 ppm of chlorine via whole-body exposure in a sealed 5-L cylindrical glass chamber (Specialty Glass Inc, Houston, Tex.). Following exposure, heart rate and SO₂p were obtained using a MouseOx (Starr Life Sciences) pulse oximeter collar. Respiratory rate (RR, bpm) was obtained manually every hour. Respiratory distress scores (0 to 6) and neuromuscular/ataxia scores (combined score of posture 0-3, tone 0-3, movement 0-2, seizure 0-2) were obtained hourly.

Scopolamine was administered 15 minutes after exposure to chlorine gas. Scopolamine was administered to test subjects in 3 ways: intramuscularly (IM, 0.15 mg/kg), intratracheally (IT, 0.2 mg/kg), or intranasally (IN or “INSCOP,” 0.16 mg/kg). The intranasal formulation was a gel as described herein.

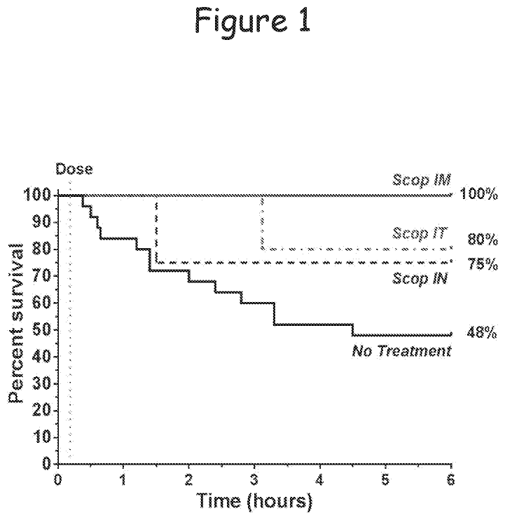

FIG. 1 is a chart showing the survival to 6 hours following exposure to high dose Cl₂. Compared to no treatment, the administration of scopolamine improved survival, with the best survival (100%) being obtained with IM dosing.

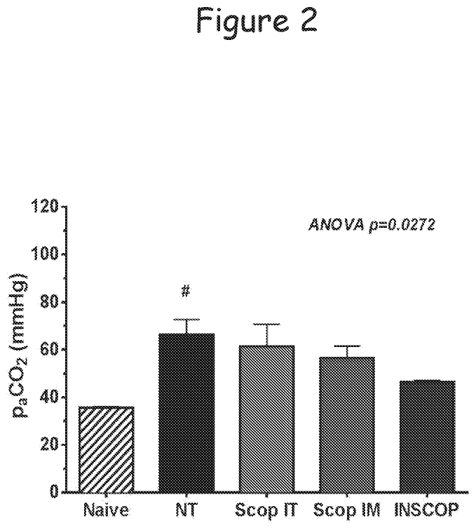

FIG. 2 is a chart showing CO₂ retention in arterial blood gas (hypercarbia) 6 hours after Cl₂ exposure. The best results (least increase in CO₂ retention) was obtained with intranasal scopolamine (as shown by the bar labeled “INSCOP”).

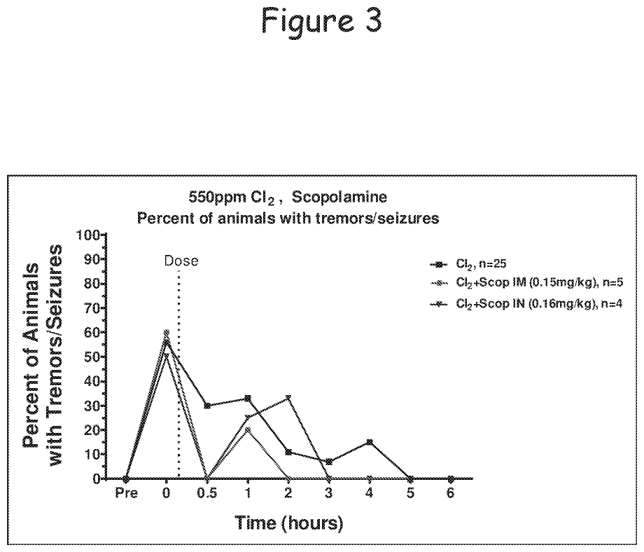

FIG. 3 is a chart showing the percent of subjects with tremors/seizures due to Cl₂ exposure. This percentage decreased after scopolamine treatment, with the best results being obtained with IM dosing.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

1-10. (canceled)

11. A method of treating exposure to chlorine gas, comprising the step of administering a therapeutically effective amount of a pharmaceutical composition comprising scopolamine to a subject in need thereof.

12. The method of claim 11, wherein the subject has a medical condition selected from the group consisting of pulmonary edema, tracheobronchitis, and acute respiratory distress syndrome.

13. The method of claim 11, wherein the composition is administered in an amount which delivers between 0.05 mg/kg and 0.3 mg/kg scopolamine to the subject

14. The method of claim 13, wherein the composition is administered in an amount which delivers between 0.1 mg/kg and 0.2 mg/kg scopolamine to the subject,

15. The method of claim 11, wherein between 0.2 mg and 10 mg scopolamine is administered to the subject.

16. The method of claim 15, wherein between 0.5 mg and 4 mg scopolamine is administered to the subject.

17. The method of claim 16, wherein between 1 mg and 2 mg scopolamine is administered to the subject.

18. The method of claim 11, wherein the scopolamine is scopolamine HBr.

19. The method of claim 11, wherein the composition is administered intranasally, intravenously, intratracheally, intramuscularly, or by inhalation.

20. The method of claim 19, wherein the composition is administered intranasally in a dose having between 0.2 mg and 2 mg scopolamine.

21. The method of claim 20, wherein the composition is administered intranasally in a dose having 0.2 mg or 0.4 mg scopolamine.

22. The method of claim 11, wherein the composition is administered within 15 minutes of exposure to chlorine gas.

23. The method of claim 11, wherein the composition is administered with an inhaler, a syringe, or an intranasal spray device.
